(19) United States
(12) Patent Application Publication
(10) Pub. No.: US 2025/0017461 A1
(43) Pub. Date: Jan. 16, 2025

(54) DIGITALLY COMBINING OVERLAY DATA AND IMAGE DATA

(71) Applicant: ALCON INC., Fribourg (CH)

(72) Inventors: Mario Abraham, Burgthann (DE); Maik Lange, Ruckersdorf (DE)

(21) Appl. No.: 18/754,267

(22) Filed: Jun. 26, 2024

Related U.S. Application Data
(60) Provisional application No. 63/512,828, filed on Jul. 10, 2023.

(51) Int. Cl. A61B 3/00; A61B 3/113; A61B 3/14; G06T 7/00; G06T 7/20; G06T 7/30; H04N 13/239; H04N 13/361

(52) U.S. Cl. CPC A61B 3/0058; A61B 3/113; A61B 3/145; G06T 7/0012; G06T 7/20; G06T 7/30; H04N 13/239; H04N 13/361; G06T 2207/10012; G06T 2207/20212; G06T 2207/30041

(57) ABSTRACT

According to certain embodiments, an ophthalmic system for providing an image of an eye includes a camera system and a computer. The camera system includes stereoscopic cameras that provide image data for images of the eye. The computer: receives the image data from the camera system; accesses overlay data for an overlay for the image of the eye; aligns the overlay data and the image data; digitally combines the overlay data and the image data to yield combined image data for an image with the overlay; and provides the combined image data to a display device to display the image with the overlay in three dimensions.

### TECHNICAL FIELD

The present disclosure relates generally to ophthalmic imaging, and more particularly to digitally combining overlay data and image data for ophthalmic imaging.

### BACKGROUND

In an ophthalmologic procedure, a physician may have to manipulate tissue of the patient's eye without injuring the eye, e.g., open or close a LASIK flap, remove a lenticule, or dissect a cataract. The physician may view the eye through a surgical microscope that provides a magnified view. In certain cases, a graphical overlay may be placed over the view to assist with the procedure.

### BRIEF SUMMARY

In certain embodiments, an ophthalmic system for providing an image of an eye includes a camera system and computer. The camera system includes stereoscopic cameras that provide image data for images of the eye. The stereoscopic cameras include a first camera and a second camera, where the first camera provides first image data for a first image and the second camera provides second image data for a second image. The computer: receives the image data from the camera system; accesses overlay data for an overlay for the image of the eye, the overlay data having first overlay data and second overlay data; aligns the overlay data and the image data; digitally combines the overlay data and the image data to yield combined image data for an image with the overlay, the overlay data and the image data combined by digitally combining the first overlay data and the first image data and digitally combining the second overlay data and the second image data; and provides the combined image data to a display device to display the image with the overlay in three dimensions.

Embodiments may include none, one, some, or all of the following features:
The ophthalmic system includes an eye tracker that detects movement of the eye from the image data. The computer realigns the overlay data and the image data in accordance with the movement.
The computer digitally combines the overlay data and the image data by combining an overlay percentage of the overlay data and an image percentage of the image data to yield the combined image data. The computer may receive user input selecting a value for the image percentage or the overlay percentage and adjust the image percentage or the overlay percentage in response to the user input. The computer may detect from the image data a change in the image of the eye and adjust the image percentage or the overlay percentage in response to the change in the image of the eye.
The computer digitally combines the overlay data and the image data by combining a first overlay percentage of the first overlay data and a first image percentage of the first image data and by combining a second overlay percentage of the second overlay data and a second image percentage of the second image data. The first overlay percentage may be substantially the same as or different from the second overlay percentage.
The overlay data includes eye tracking information to yield an eye position overlay that indicates the position of the eye.
The overlay data includes diagnostic information to yield a diagnostic overlay that describes the eye.
The overlay data includes treatment information to yield a treatment overlay that describes a treatment for the eye.

In certain embodiments, an ophthalmic system for providing an image of an eye includes a camera system, eye tracker, and computer. The camera system includes one or more cameras that provide image data for images of the eye, where each camera provides the image data for an image. The eye tracker determines the location of the eye according to the image data. The computer: receives the image data from the camera system; accesses overlay data for an overlay for the image of the eye; aligns the overlay data and the image data in accordance with the location of the eye; digitally combines the overlay data and the image data to yield combined image data for an image with the overlay; and provides the combined image data to a display device configured to display the image with the overlay.

Embodiments may include none, one, some, or all of the following features:
The eye tracker detects movement of the eye from the image data, and the computer realigns the overlay data and the image data in accordance with the movement.
The computer digitally combines the overlay data and the image data by combining an overlay percentage of the overlay data and an image percentage of the image data to yield the combined image data. The computer may detect from the image data a change in the image of the eye and adjust the image percentage or the overlay percentage in response to the change in the image of the eye.
The camera system includes stereoscopic cameras comprising a first camera and a second camera, where the first camera provides first image data for a first image and the second camera provides second image data for a second image. The overlay data includes first overlay data and second overlay data. The computer digitally combines the overlay data and the image data by combining the first overlay data and the first image data; and combining the second overlay data and the second image data.
The overlay data includes eye tracking information to yield an eye position overlay that indicates the position of the eye.
The overlay data includes diagnostic information to yield a diagnostic overlay that describes the eye.
The overlay data includes treatment information to yield a treatment overlay that describes a treatment for the eye.

### BRIEF DESCRIPTION OF THE DRAWINGS

FIG. 1 illustrates an example of an ophthalmic system that digitally inserts a graphical overlay into an image of an eye, according to certain embodiments;

FIG. 2 illustrates an example of the camera system of FIG. 1, according to certain embodiments;

FIG. 3 illustrates an example of an ophthalmic system that provides a three-dimensional (3D) overlay in a 3D image, according to certain embodiments;

FIGS. 4A through 6B illustrate examples of overlays aligned with an eye image, according to certain embodiments: FIGS. 4A and 4B illustrate an example of a diagnostic overlay of measured corneal pachymetry;

FIG. 5 illustrates an example of a treatment overlay of a planned refractive or cataract treatment; and FIGS. 6A and 6B illustrate an example of a treatment overlay that shows markings for a lenticule and associated incisions; and

FIG. 7 illustrates an example of a method for providing an image with an overlay, according to certain embodiments, which may be performed by the ophthalmic system of FIG. 1.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

Known techniques for inserting an overlay into an image of the eye use optical elements, such as splitter mirrors, to optically combine light transmitting the eye image with the light transmitting the overlay. However, mirrors restrict transmission of the light, reducing the contrast in the resulting image. Moreover, aligning the overlay with the eye image with mirrors can be difficult.

The systems described in this document provide two-dimensional (2D) and/or three-dimensional (3D) digital overlays to the display device of a digital microscope. In contrast to an optical overlay, the system digitally inserts the overlay information into the image, like in augmented reality. The overlay information is inserted at the proper locations of the eye image to align the overlay with the eye. Moreover, in response to movement detected by an eye tracker, the system moves the overlay relative to the eye image to realign the overlay with the eye. The overlays may assist with ophthalmic diagnosis, treatment (planning and/or implementation), and/or training.

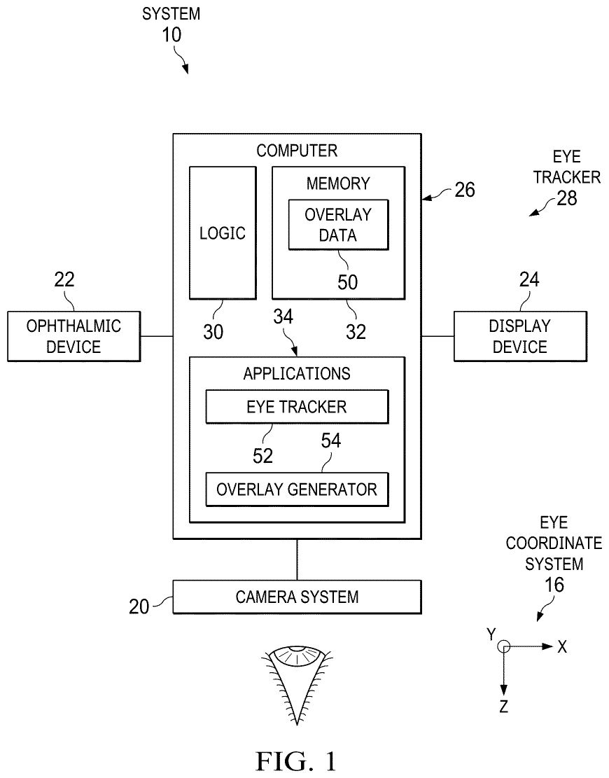

FIG. 1 illustrates an example of an ophthalmic system 10 that digitally inserts a graphical overlay into an image of an eye, according to certain embodiments. Ophthalmic system 10 includes a camera system 20, an ophthalmic device 22, a display device 24, and a computer 26, coupled as shown. Computer 26 include logic 30, memory 32, and applications 34. Memory stores overlay data 50, and applications 34 include eye tracker 52 and overlay generator 54 applications. An eye tracker 28 includes camera system 20 and eye tracker 52 application.

For case of explanation, certain eye features may be used to define an example coordinate system 16 (x, y, z) of the eye. For example, the eye has a center (e.g., pupil center, limbus center, apex, or vertex) and an eye axis (e.g., optical or pupillary axis) that can define the z-axis of eye coordinate system 16, which in turn defines an xy-plane of system 16. In addition, a position A relative to B may describe the distance and/or the orientation between A and B. For example, the position of a camera relative to the eye may be the distance between the camera and the eye and/or the direction of the camera axis relative to the eye axis.

As an overview, camera system 20 includes cameras that provide image data for images of the eye. Eye tracker 28 determines the location of the eye according to the image data. Computer 26 receives image data from camera system 20 and accesses overlay data for an overlay for the eye. Computer 26 aligns the overlay data and the image data in accordance with the location of the eye, and digitally combines the overlay data and the image data to yield combined image data for an image with the overlay. Computer 26 provides the combined image data to display device 24 to display the image (in 2D or 3D) with the overlay. In addition, computer 26 realigns the overlay data and the image data in response to movement detected by eye tracker 28.

Turning to the components of the example, camera system 20 includes cameras. A camera detects light from an object and provides a signal with image data that can be used to generate images of the eye. The image data are provided to computer 26 for eye tracking (and optionally other analysis) and to display device 24 to present the images of the eye. Examples of cameras include a charged-coupled device (CCD), video, complementary metal-oxide semiconductor (CMOS) sensor (e.g., active-pixel sensor (APS)), line sensor, and optical coherence tomography (OCT) camera.

A camera detects light of any suitable spectral range, e.g., a range of infrared (IR), ultraviolet (UV), and/or visible (VIS) wavelength light, where a range can include a portion or all of the wavelength. For example, a camera may detect visible light, infrared light, or other visible and infrared light from the eye to yield an image. Certain cameras may capture features of the eye (e.g., e.g., pupil, iris, blood vessels, limbus, sclera, eyelashes, and/or eyelid) better than others. For example, an infrared camera generally provides more stable pupil tracking and better contrast for iris structures. A visible range camera yields better images of blood vessels. Accordingly, an IR camera may be used to monitor lateral movement by tracking the pupil and/or or to monitor cyclotorsion by tracking iris structures. A visible range camera may be used to monitor translation and/or rotational movement by tracking blood vessels.

A camera may record images at any suitable frequency or resolution. A higher speed camera may record images at greater than, e.g., 400 to 600 frames per second, such as greater than 500 frames per second. A higher resolution camera may yield images with greater than, e.g., 4 to 6 megapixels, such as greater than 5 megapixels. In general, higher resolution images and higher speed image acquisition may provide more accurate tracking, but they both may require more computing time to process, so there may be a trade-off between resolution and speed. Accordingly, the speed and/or resolution of a camera may be selected for particular purposes. In certain embodiments, a higher speed camera may track eye features that move faster and/or can be identified with lower resolution, and a higher resolution camera may be used to track eye features that require higher resolution for identification and/or move more slowly. For example, a lower resolution, higher speed camera may track the pupil (which does not require high resolution) to detect xy movement. As another example, a higher resolution, lower speed camera may track blood vessels/iris structures to detect rotations, z-movement.

Ophthalmic device 22 may be a system that is used to diagnose and/or treat an eye. Examples include a refractive surgical system, a cataract system, a topographer, an OCT measuring device, and a wavefront measuring device. Display device 24 provides images, e.g., the combined image, to the user of system 10. Examples of display device 24 include a computer monitor, a 3D display, a projector/beamer, a TV monitor, binocular displays, glasses with monitors, a virtual reality display, and an augmented reality display.

Computer 26 controls components of system 10 (e.g., camera system 20, ophthalmic device 22, display device 24, and/or eye tracker 28) and uses overlay generator 54 to generate eye images with overlays. In general, computer 26 uses eye tracker 28 and eye tracker application 52 to track the position (e.g., location and/or orientation) of an eye. Computer 16 receives image data from camera system 20, aligns the overlay and image data in accordance with the location of the eye, and digitally combines the image data with overlay data to yield an image with an overlay.

In certain embodiments, the overlay may be designed to be positioned (located and/or orientated) relative to one or more features of the eye or other suitable body part of the patient. Computer 26 determines the location of the feature(s) in the image data and positions (e.g., sets the location and/or orientation) of the overlay relative to the feature(s) as designed. For example, the center point of the overlay may be designed to be located at the center of the eye, so computer 26 determines the eye center in the image data and positions the overlay center point at the eye center. As another example, multiple points of the overlay may be designed to be located and oriented relative to multiple eye features (e.g., iris, pupil, or sclera features), so computer 26 identifies the eye features in the image data and position the overlay points relative to the features. As yet another example, one or more points of the overlay may be designed to be located and/or oriented relative to one or more points of any suitable body part of the patient, e.g., the eyelashes, eye boundary, nose, or other facial feature. In certain embodiments, computer 26 realigns an overlay in response to movement of the eye as detected by, e.g., eye tracker 28, such that the overlay remains in the proper position relative to the eye feature(s).

After aligning the data, computer 26 digitally combines the overlay and image data to yield combined image data that displays the image with the overlay. For pixels where the overlay is present, computer 26 may adjust the pixels such that they display the overlay superimposed over the eye image. Pixel data may include light and/or color information for a pixel. An overlay may include transparent, translucent, and/or opaque portions. For a transparent portion of the overlay, computer 26 may use the pixel data only from the image data such that only the eye image is displayed at that portion. For an opaque portion, computer 26 may use the pixel data only from the overlay data such that only the overlay is displayed at that portion.

For a translucent portion of the overlay, computer 26 may use any suitable combination of pixel data from the image and overlay data such that a combination of the eye image and the overlay is displayed at that portion. For example, computer 26 may combine a percentage of the pixel data of the image data (“image percentage”) and a percentage of the pixel data of the overlay data (“overlay percentage”). The image percentage Pi and overlay percentage Po may have any suitable values. For example, Pi+Po may equal 100%, where Pi=Po, Pi>Po, or Pi<Po. Generally, a higher image percentage and/or lower overlay percentage displays a more visible eye image relative to the overlay, and a higher overlay percentage and/or lower image percentage displays a more visible overlay relative to the eye image. For example, an overlay percentage Po of 40 percent or less (such as 30 to 20 or 20 to 10 percent) may yield a translucent overlay.

The image and overlay data may be combined in any suitable manner. In certain embodiments, the image and overlay percentages may be predefined and/or may be adjusted in response to input from the user to select a more visible image or overlay. In certain embodiments, computer 26 may detect from the image data a change in pixels values and in response may automatically adjust the image and overlay percentages. For example, computer 26 may detect from the image data that the eye image is suddenly brighter, and decrease the image percentage to allow the overlay to still be visible. Conversely, computer 26 may detect that the eye image is suddenly darker, and increase the image percentage to allow the eye image to still be visible.

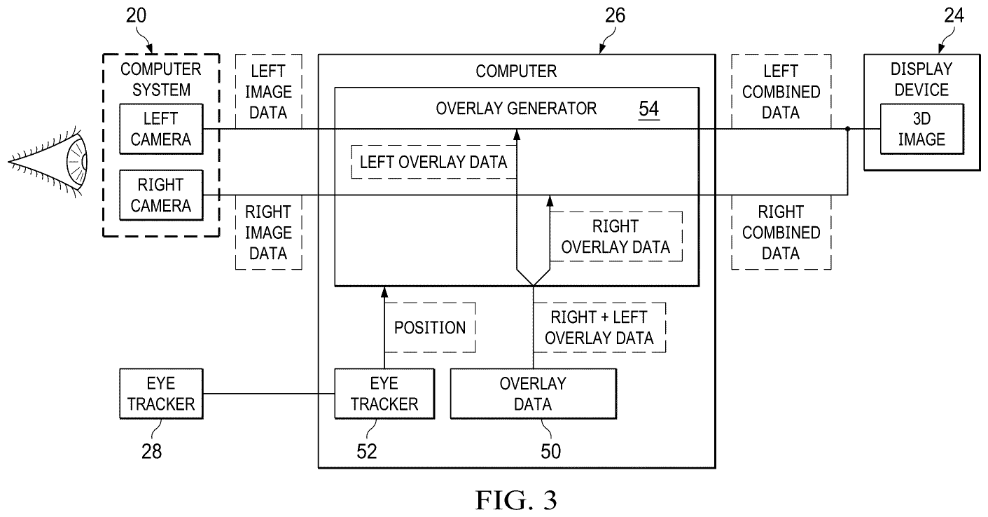

In certain embodiments, as described in FIG. 3, system 10 may provide image data for a 3D overlay in a 3D image. In these embodiments, computer 26 may use the same or different image and overlay percentages for the left and right image data. Using the same percentages yields substantially the same relative visibility of the eye image and overlay in the left and right images. Using different percentages yields different relative visibilities in the left and right images, e.g., the eye image may be more visible in the left image and the overlay may be more visible in the right image, which may compensate for the user's vision issues.

Computer 26 provides the combined image data to display device 24 to display the 3D image with the 3D overlay. The 3D images may be provided in any suitable manner. For example, the combined image data may be provided in left and right displays or may be provided intermingled in one display.

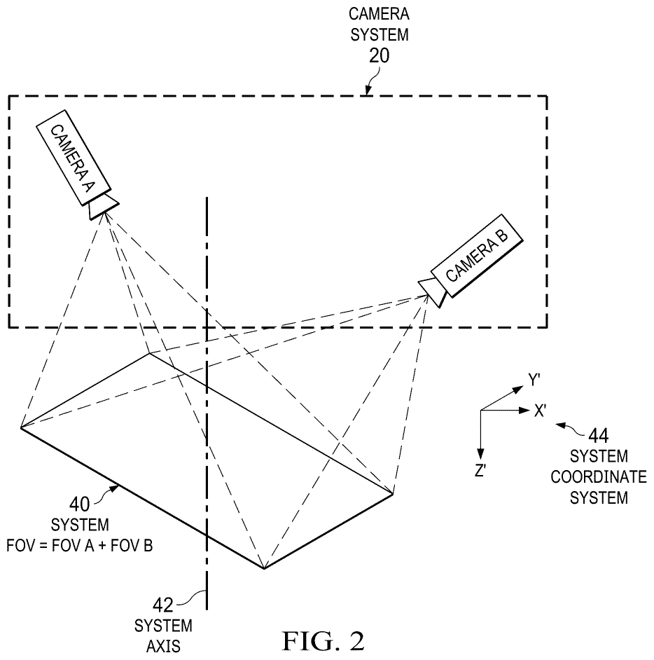

FIG. 2 illustrates an example of camera system 20 of FIG. 1, according to certain embodiments. Camera system 20 includes Camera A with a field of view (FOV) A and Camera B with FOV B, where both FOV A and FOV B extend over system FOV 40. The cameras of camera system 20 may have any suitable arrangement. In the example, Camera A and Camera B are arranged mirror symmetrically about a system axis 42. The images may be stercoscopically reconstructed to yield a 3D image of an eye.

In the example, camera system 20 has a system FOV 40, a system axis 42, and a system coordinate system 44 (x′, y′, z′). In the example, system FOV 40 includes the FOVs of cameras A and B. System axis 42 may have any suitable position, e.g., axis 42 may be substantially orthogonal to system FOV 40 and may pass through the center of system FOV 40. In the example, system axis 42 defines the z′-axis of system coordinate system 44.

Computer 26 aligns and combines image portions to yield a combined image. The image portions may be aligned in any suitable manner. For example, each camera has a known position, such as a location (e.g., distance away from system FOV 40 or eye region 14) and orientation (e.g., camera optical axis relative to system axis 42 or eye axis 15). From this information, computer 26 can determine the positions of the image portions to align them within the combined image. As another example, the cameras each generate an image of a calibration figure (e.g., a checkerboard), and the positions of the cameras are determined from the images. As yet another example, a user calibrates the image portions by manually aligning the portions when viewed through the cameras. Computer 26 records the positions of the aligned portions.

FIG. 3 illustrates an example of an ophthalmic system 10 that provides a three-dimensional (3D) overlay in a 3D image, according to certain embodiments. Image portions may be combined to yield a two-dimensional (2D) image with a 2D overlay, or image portions (e.g., from stereoscopic cameras) may be combined to yield a three-dimensional (3D) image with a 3D overlay. 3D overlays may provide important z-direction information. For example, a 3D overlay can provide the depth (2-direction) profile of a lenticule pattern. As another example, a 3D overlay can show the corneal topography in the z-direction, which may be important when, e.g., cutting a LASIK flap.

In the example, camera system 20 includes stereoscopic cameras comprising first and second cameras, which may be any suitable system of cameras, such as a left (L) camera and a right (R) camera (as shown in the example) or an upper and lower camera. In the example, the left camera provides left image data for a left image, and the right camera provides right image data for a right image. Overlay data 50 includes left overlay data and right overlay data. Eye tracker 28 and eye tracker application 54 provides the position of the eye to computer 26. Overlay generator 54 uses the eye tracker information to align the overlay and image data. Overlay generator 54 then digitally combines the overlay data and the image data by combining the left overlay data and the left image data and combining the right overlay data and the right image data. The left and right combined data is sent to display device 24 to generate a 3D image with a 3D overlay.

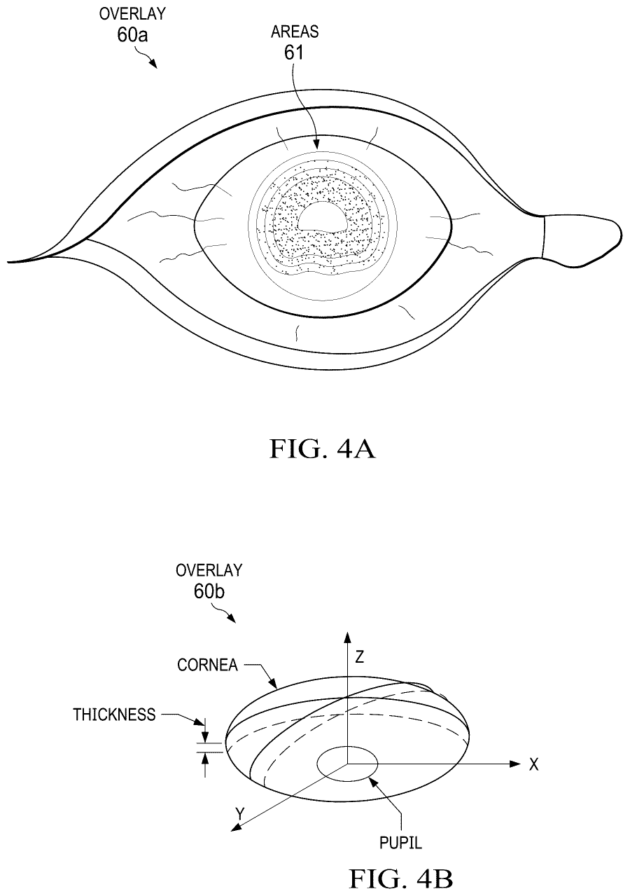

FIGS. 4 through 6 illustrate examples of overlays 60 (a, b, c) aligned with an eye image, according to certain embodiments. An overlay may be any suitable shape, size, or color. For example, an overlay may be substantially the same size as a feature of the eye (e.g., pupil, iris, and/or sclera) and may include colors that contrast with the features of the eye. Parts of the overlay may be transparent, translucent, and/or opaque.

An overlay may be static or move with the eye. For example, in response to eye tracking information describing movement of the eye, the overlay may be adjusted in accordance with the movement. An overlay may be displayed at any suitable time of the procedure. For example, prior to starting a procedure, the treatment profile and/or predicted outcome may be displayed as overlays. An overlay may include any suitable information. Examples of such information is described in the following.

Eye Position Overlay. An eye position overlay indicates the position of the eye and may be generated from overlay data comprising eye tracking information from the eye tracker. The overlay may include markings indicating the position of, e.g., the pupil, limbus, vertex, or other feature(s) of the eye. This overlay may be used to, e.g., position the system, align a treatment profile, calibrate the system, or verify the eye tracking system.

Diagnostic Overlay. A diagnostic overlay describes the eye and may be generated from overlay data comprising diagnostic information, such known or measured information about the eye. Examples of such information include: biometric measurements (e.g., the anterior chamber depth, eye length, corneal thickness, and/or crystalline lens thickness); diagnostic profile (e.g., a corneal topography, local pachymetry, and/or local refraction); or a map of tissue irregularities. The diagnostic information may be presented in any suitable manner. For example, a color overlay may include different colors that indicate different thicknesses, depths, or tissue irregularities. As another example, thickness and/or depth may be represented by point grids (e.g., distance between points indicate thickness/depth); mesh (e.g., meridians or a web); elevation lines; distance vectors or lines orthogonal to a surface; a 3D solid graphical object; or other suitable representation.

FIGS. 4A and 4B illustrate examples of diagnostic overlays 60a and 60b of measured corneal pachymetry. Overlay 60a shows different areas 61 of the eye, where each area 61 represents a different thickness or range of thicknesses. Each area 61 may be distinguished from an adjacent area 61 by, e.g., color, grey value, translucency, a border, or other marking. Overlay 60b is a 3D graphical element that shows the thickness of the cornea.

Treatment Overlay. A treatment overlay describes a treatment for the eye and may be generated from overlay data comprising treatment information. A treatment overlay may include markings for incisions or insertions. Markings for incisions include, e.g., markings for a LASIK flap to be cut, an existing flap, corneal channels, or lenticule. Markings for insertions include, e.g., markings for a Kamra inlay, artificial lens, implantable collamer lens (ICL), or keratoplastic inlay.

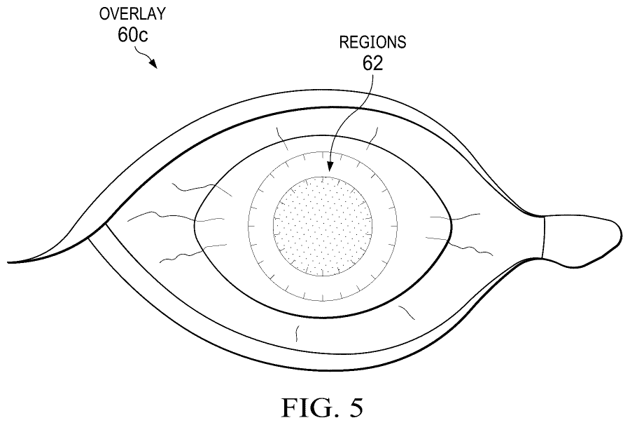

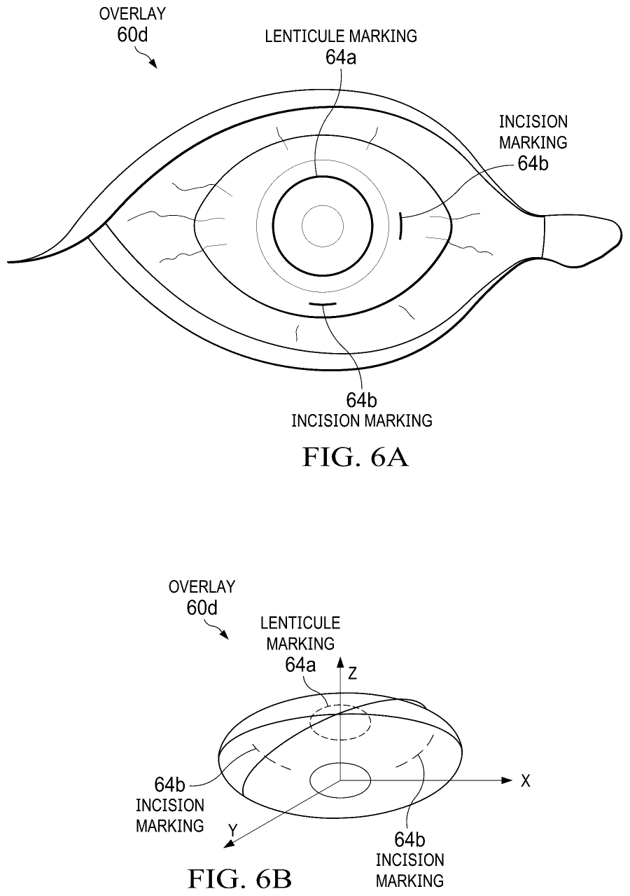

FIGS. 5 and 6 illustrate examples of treatment overlays 60c-d. FIG. 5 illustrates an example of a treatment overlay 60c of a planned refractive or cataract treatment. The different regions 62 correspond to different ablation depths. FIGS. 6A and 6B illustrate other examples of a treatment overlay 60d that shows markings 64 (64a, 64b) for a lenticule and associated incisions. FIG. 6A is a top view and FIG. 6B is a perspectival 3D view of lenticule marking 64a and incision markings 64b. Note that in FIG. 6B, markings 64 appear under the corneal surface in the 3D image. In other examples, lenticule marking 64a may be replaced by a 3D rendering of the lenticule in the 3D image. The 3D lenticule may better show to a user the thicknesses between the anterior surface of the lenticule and the anterior corneal surface and between the posterior surface of the lenticule and the posterior corneal surface.

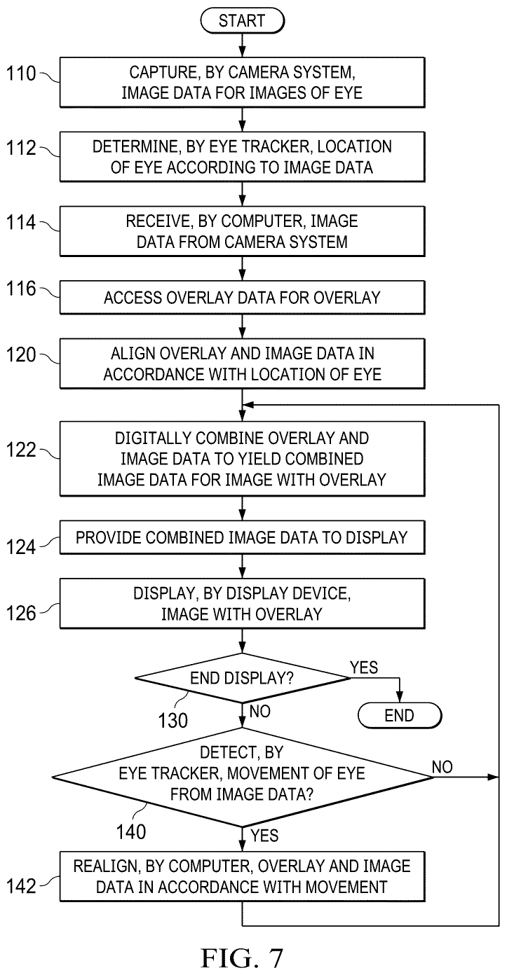

FIG. 7 illustrates an example of a method for providing an image with an overlay, according to certain embodiments. The method may be performed by ophthalmic system 10 of FIG. 1. The method starts at step 110, where camera system 20 captures image data for images of an eye.

Eye tracker 28 determines the location of the eye according to the image data at step 112. Computer 26 receives the image data from camera system 20 at step 114, and accesses overlay data for an overlay at step 116. Computer 26 aligns the overlay and image data in accordance with the location of the eye at step 120, and digitally combines the overlay and image data at step 122 to yield combined image data for the image with the overlay. In certain embodiments that provide a 3D image, computer 26 may combine the overlay and image data by combining left overlay data and left image data to yield a left image and by combining right overlay data and right image data to yield a right image. The combined image data is provided to display device 24 at step 124, which displays the 3D image with the 3D overlay at step 126.

The display of eye images may end at step 130. If the display is to continue, the method proceeds to set 140, where eye tracker 28 may detect movement of the eye from the image data. If movement is detected, the method proceeds to step 142, where computer 26 realigns the overlay and image data in accordance with the movement. The method then returns to step 122, where computer 26 digitally combines the overlay and image data to yield combined image data for an adjusted image with the overlay. If movement is not detected, the method returns to step 122. If the method is to end at step 130, the method ends.

A component (such as the control computer) of the systems and apparatuses disclosed herein may include an interface, logic, and/or memory, any of which may include computer hardware and/or software. An interface can receive input to the component and/or send output from the component, and is typically used to exchange information between, e.g., software, hardware, peripheral devices, users, and combinations of these. A user interface is a type of interface that a user can utilize to communicate with (e.g., send input to and/or receive output from) a computer. Examples of user interfaces include a display device, Graphical User Interface (GUI), touchscreen, keyboard, mouse, gesture sensor, microphone, and speakers.

Logic can perform operations of the component. Logic may include one or more electronic devices that process data, e.g., execute instructions to generate output from input. Examples of such an electronic device include a computer, processor, microprocessor (e.g., a Central Processing Unit (CPU)), and computer chip. Logic may include computer software that encodes instructions capable of being executed by an electronic device to perform operations. Examples of computer software include a computer program, application, and operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database, network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. § 112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112(f).

What is claimed:

1. An ophthalmic system for providing an image of an eye, comprising:
a camera system comprising a plurality of stereoscopic cameras configured to provide image data for one or more images of the eye, the stereoscopic cameras comprising a first camera and a second camera, the first camera configured to provide first image data for a first image, the second camera configured to provide second image data for a second image; and
a computer configured to:
receive the image data from the camera system;
access overlay data for an overlay for the image of the eye, the overlay data comprising first overlay data and second overlay data;
align the overlay data and the image data;
digitally combine the overlay data and the image data to yield combined image data for an image with the overlay, the overlay data and the image data combined by digitally combining the first overlay data and the first image data and combining the second overlay data and the second image data; and
provide the combined image data to a display device configured to display the image with the overlay in three dimensions.

2. The ophthalmic system of claim 1:
further comprising an eye tracker configured to detect movement of the eye from the image data; and
the computer configured to realign the overlay data and the image data in accordance with the movement.

3. The ophthalmic system of claim 1, the computer configured to digitally combine the overlay data and the image data by:
combining an overlay percentage of the overlay data and an image percentage of the image data to yield the combined image data.

4. The ophthalmic system of claim 3, the computer configured to:
receive user input selecting a value for the image percentage or the overlay percentage; and
adjust the image percentage or the overlay percentage in response to the user input.

5. The ophthalmic system of claim 3, the computer configured to:
detect from the image data a change in the image of the eye; and
adjust the image percentage or the overlay percentage in response to the change in the image of the eye.

6. The ophthalmic system of claim 1, the computer configured to digitally combine the overlay data and the image data by:
combining a first overlay percentage of the first overlay data and a first image percentage of the first image data; and
combining a second overlay percentage of the second overlay data and a second image percentage of the second image data.

7. The ophthalmic system of claim 6, the first overlay percentage substantially the same as the second overlay percentage.

8. The ophthalmic system of claim 6, the first overlay percentage different from the second overlay percentage.

9. The ophthalmic system of claim 1, the overlay data comprising eye tracking information to yield an eye position overlay that indicates the position of the eye.

10. The ophthalmic system of claim 1, the overlay data comprising diagnostic information to yield a diagnostic overlay that describes the eye.

11. The ophthalmic system of claim 1, the overlay data comprising treatment information to yield a treatment overlay that describes a treatment for the eye.

12. An ophthalmic system for providing an image of an eye, comprising:
a camera system comprising one or more cameras configured to provide image data for one or more images of the eye, each camera configured to provide the image data for an image of the one or more images of the eye;
an eye tracker configured to determine a location of the eye according to the image data; and
a computer configured to:
receive the image data from the camera system;
access overlay data for an overlay for the image of the eye;
align the overlay data and the image data in accordance with the location of the eye;
digitally combine the overlay data and the image data to yield combined image data for an image with the overlay; and
provide the combined image data to a display device configured to display the image with the overlay.

13. The ophthalmic system of claim 12:
the eye tracker configured to detect movement of the eye from the image data; and
the computer configured to realign the overlay data and the image data in accordance with the movement.

14. The ophthalmic system of claim 12, the computer configured to digitally combine the overlay data and the image data by:
combining an overlay percentage of the overlay data and an image percentage of the image data to yield the combined image data.

15. The ophthalmic system of claim 14, the computer configured to:
detect from the image data a change in the image of the eye; and
adjust the image percentage or the overlay percentage in response to the change in the image of the eye.

16. The ophthalmic system of claim 12:
the camera system comprising a plurality of stereoscopic cameras comprising a first camera and a second camera, the first camera configured to provide first image data for a first image, the second camera configured to provide second image data for a second image;
the overlay data comprising first overlay data and second overlay data; and
the computer configured to digitally combine the overlay data and the image data by:
combining the first overlay data and the first image data; and
combining the second overlay data and the second image data.

17. The ophthalmic system of claim 12, the overlay data comprising eye tracking information to yield an eye position overlay that indicates a position of the eye.

18. The ophthalmic system of claim 12, the overlay data comprising diagnostic information to yield a diagnostic overlay that describes the eye.

19. The ophthalmic system of claim 12, the overlay data comprising treatment information to yield a treatment overlay that describes a treatment for the eye.
